(19) United States
(12) Patent Application Publication
(10) Pub. No.: US 2016/0051526 A1
(43) Pub. Date: Feb. 25, 2016

(54) The Local Treatment of Inflammatory Ophthalmic Disorders

(71) Applicant: CRESSET BIOMOLECULAR DISCOVERY LTD, Welwyn Garden City Hertfordshire (GB)

(72) Inventors: Alan Leslie Rothaul, Oxfordshire (GB); Jeremy Gilbert Vinter, Hertfordshire (GB); Robert Arthur Scoffin, Cambridgeshire (GB)

(21) Appl. No.: 14/783,038

(22) PCT Filed: Apr. 9, 2014
(86) PCT No.: PCT/GB2014/051108

§ 371 Date: Oct. 7, 2015

(30) Foreign Application Priority Data
Apr. 9, 2013 (GB) 1306411.8
Apr. 9, 2013 (GB) 1306413.4

(51) Int. Cl. A61K 31/4375; A61K 45/06; A61K 31/56

(52) U.S. Cl. CPC A61K 31/4375; A61K 31/56; A61K 45/06

(57) ABSTRACT

The present invention provides Nalidixic acid and analogues of Nalidixic acid, pharmaceutical compositions including at least one of Nalidixic acid and analogues of Nalidixic acid, and methods for treating inflammatory ophthalmic disorders by local administration. The ophthalmic disorders may be characterized by ocular inflammation, dry eye disorders, pathological ocular angiogenesis, or retinal or sub-retinal edema.

### FIELD OF THE INVENTION

This invention relates to the local use of Nalidixic acid and Nalidixic acid analogues for the treatment of inflammatory ophthalmic diseases characterized by ocular inflammation, dry eye disorders, pathologic ocular angiogenesis and/or retinal or sub-retinal edema.

### BACKGROUND OF THE INVENTION

Dry eye, or keratoconjunctivitis, is a common ophthalmological disease affecting millions of people each year, it is reported to have an overall prevalence of between 5% and 6% of the population, with frequency of occurrence increasing with age. The condition is particularly prevalent in post-menopausal women due to hormonal changes caused by the cessation of fertility. Dry eye is primarily caused by the break-down of the pre-ocular tear film which results in dehydration of the exposed outer surface. There is a strong rationale that ocular inflammation as a result of pro-inflammatory cytokines and growth factors plays a major role in the underlying causes of dry eye. As such, locally administered anti-cytokine or general anti-inflammatory agents are often used in the treatment of dry eye. Other forms of conjunctivitis are also poorly treated; allergic conjunctivitis only responds poorly to standard topical anti-allergy treatment while viral and bacterial conjunctivitis often require long term treatment with anti-infectives or antibiotics.

Another disease of the interior of the eye is uveitis, or inflammation of the uveal tract. The uveal tract (uvea) is composed of the iris, ciliary body and choroid. Uveitis may be caused by trauma, infection or surgery and can affect any age group. The disease is classified anatomically as anterior, intermediate, posterior or diffuse. Anterior uveitis affects the anterior portion of the eye including the iris. Intermediate uveitis, also called peripheral uveitis, is centred in the area immediately behind the iris and lens in the region of the ciliary body. Posterior uveitis may also constitute a form of retinitis, or it may affect the choroids and the optic nerve. Diffuse uveitis involves all parts of the eye. The most common treatment of uveitis is with locally administered glucocorticosteroids often in combination with other anti-inflammatory drugs. Although these drugs are effective in the treatment of many forms of ocular inflammation they have several side-effects including endophthalmitis, cataracts and elevated intra-ocular pressure (IOP). There is a need for potent anti-inflammatory agents with an improved side effect profile, the so called non-steroid steroid, for the treatment of ophthalmic inflammation and edema.

Diseases and degenerative conditions of the optic nerve and retina are the leading causes of blindness in the world. A significant degenerative condition of the retina is age-related macular degeneration (ARMD). ARMD is the most common cause of blindness in people over 50 in the USA and its prevalence increases with age. ARMD is classified as either wet (neovascular) or dry (non-neovascular) where the dry form of the disease is the most common. Macular degeneration occurs when the central retina has become distorted and thinned usually associated with age but also characterised by intra-ocular inflammation and angiogenesis (wet ARMD only) and/or intra-ocular infection.

Retinopathy associated with diabetes is a leading cause of blindness in type I diabetes and is also common in type II diabetes. The degree of retinopathy depends on the duration of diabetes and generally begins to occur ten or more years after onset of diabetes. Diabetic retinopathy may be classified as non-proliferative, where the retinopathy is characterised by increased capillary permeability, edema and exudates, or proliferative, where the retinopathy is characterised by neovascularisation extending from the retina to the vitreous humor, scarring, deposit of fibrous tissue and the potential for retinal detachment. Diabetic retinopathy is believed to be caused by the development of glycosylated proteins due to high blood glucose. The subsequent generation of free-radicals, resulting in oxidative tissue damage, local inflammation and production of growth factors (such as VEGF and FGF) and inflammatory mediators, leads to inappropriate neovascularisation in common with the wet form of ARMD. Several other less common retinopathies include choroidal neovascular membrane (CNVM), cystoid macular edema (CME), epiretinal membrane (ERM) and macular hole. Today, no drugs are approved for the treatment of diabetic retinopathy or macular edema. The current standard treatment is laser photocoagulation which by destroying local tissue, decreases the production of cytokines and growth factors, but is unfortunately cytodestructive and causes permanent impairment of vision. These neovascular diseases have the potential to be treated with angiostatic agents alone or in combination with anti-inflammatory drugs.

Refractive eye surgery is any eye surgery used to improve the refractive state of the eye and thus decrease or eliminate dependency on glasses and contact lenses. This can be taken to include surgical remodelling of the cornea or cataract surgery. Successful refractive eye surgery can reduce or eliminate common vision disorders such as myopia, hyperopia and astigmatism. Common procedures for refractive eye surgery include: Flap techniques in laser ablation, performed under a partial thickness corneal flap (e.g. Laser Assisted In-Situ Keratomileusis-LASIK); Surface procedures, in which a laser is used to ablate the most anterior portion of the corneal stroma, which do not require a partial thickness cut of the corneal stroma, e.g. Photoreactive Keratectomy (PRK) and Laser Assisted Sub-Epithelium Keratomileusis (LASEK); Corneal incision procedures e.g. radial keratotomy, arcuate keratotomy and limbal relaxing incisions. Following refractive eye surgery localised inflammation at the site of surgery is common and topical and or systemic anti-inflammatory drugs, for example systemic ibuprofen and or topical glucocorticosteroids are commonly administered. In addition, dry-eye or keratoconjunctivitis may occur after refractive eye surgery. This may be temporary or permanent in nature.

Annexin-A1 (Lipocortin-1) is a 36 kDa protein which was first described in the late 1970's. It is found in many cell types and is known to play a key role in modulating the anti-inflammatory activity of exogenous and endogenous glucocorticosteroids. Annexin-Al enhances the anti-inflammatory activity of steroids and in Annexin-Al knock-out mice steroids are ineffective in animal inflammation models while Annexin-Al itself is effective in animal models of inflammation (Perretti M. and Dalli J. British Journal of Pharmacology (2009) 158, p 936-946).

Inactive Annexin-Al is released intracellularly by the nuclear action of glucocorticoid receptor stimulation. It is translocated to the cell membrane where it is phosphorylated by protein kinase C and released as an anti-inflammatory protein. The phosphatase PP2A is responsible for deactivating the anti-inflammatory activity of Annexin-A1 by direct de-phosphorylation and deactivation of protein kinase C (Yazid S. et al. Pharmacological Reports (2010) 62, p 511-517). It is hypothesised that an inhibitor of PP2A would provide a potent anti-inflammatory agent.

### SUMMARY OF THE INVENTION

The present invention relates to the use of Nalidixic acid and analogues of Nalidixic acid, by local administration, in the treatment of inflammatory ophthalmic conditions.

Surprisingly it has been found that Nalidixic acid (I) and some analogues of Nalidixic acid are effective at treating inflammatory conditions of the eye.

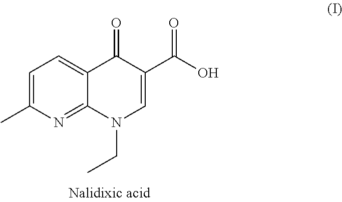 [SMILES: CCn1cc(C(=O)O)c(=O)c2ccc(C)nc21]

It has been found that Nalidixic acid and some analogues are potent inhibitors of the phosphatase PP2A thereby enhancing the anti-inflammatory activity of endogenous Annexin-A1. Nalidixic acid is an antibiotic most often used to treat urinary tract infections because it is rapidly excreted by the renal route and therefore has poor systemic pharmacokinetics. Typically this agent requires four times daily treatment by the oral route of administration to achieve anti-bacterial activity. It has now been found that the use of Nalidixic acid or a Nalidixic acid analogue or a pharmaceutically acceptable salt thereof is effective in the treatment of inflammatory ophthalmic diseases such as, but not limited to those described above.

Thus, according to the present invention, an inflammatory ophthalmic disease as described above is treated by local administration of a compound of formula (I), an analogue of formula (II) or a pharmaceutically acceptable salt thereof.

### DESCRIPTION OF THE FIGURES

FIG. 1 represents the % net histamine release from human mast cells by Nalidixic acid.

FIG. 2 represents the inhibition of Prostaglandin D2 release from human mast cells by Nalidixic acid.

FIG. 3 represents the release of Annexin-Al from human mast cells in response to increasing concentrations of Nalidixic acid.

FIG. 4 represents the reduction in clinical scores by Nalidixic Acid in a murine model of allergic conjunctivitis.

FIG. 5 represents the reduction in neutrophil invasion into retinal tissue by Nalidixic Acid in a murine model of uveitis.

### DETAILED DESCRIPTION OF THE INVENTION

Local administration of Nalidixic acid (1), or a pharmaceutically acceptable salt of Nalidixic acid to the eye is useful for the treatment of a range of ophthalmic conditions such as ocular inflammation, dry eye disorders, pathological ocular angiogenesis and retinal or sub-retinal edema.

According to another aspect of the present invention local administration of a compound of general formula (II)

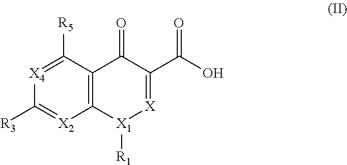

wherein,
X and X₁ independently represent CH or N;
X₂ represents C(R₂) or N;
X₄ represents C(R₄) or N;
R₁ is H, CF₃, CONH₂, CN, halogen, NH₂, NH-alkyl, alkyl, cycloalkyl or phenyl and is optionally substituted with one or more R₆; wherein R₁ may form part of a cycle with R₂;
R₂ is H, CF₃, CONH₂, CN, halogen, NH₂, alkyl, O-alkyl or S-alkyl; wherein R₂ may form part of a cycle with R₁, wherein the cycle is a 5-membered or 6-membered saturated or unsaturated cycle containing one or more atoms selected from C, N, S and O;
R₃ is H, CF₃, CONH₂, CN, halogen, NH₂, alkyl, O-alkyl, pyridyl, cycloalkyl or heterocycloalkyl and is optionally substituted with one or more R₆; wherein R₃ may form part of a cycle with R₄;
R₄ is H, F or O-alkyl; wherein R₄ may form part of a cycle with R₃, wherein the cycle is a 5-membered or 6-membered saturated or unsaturated cycle containing one or more atoms selected from C, N, S and O;
R₅ is H, F, Cl, alkyl, O-alkyl or NH₂;
R₆ is F, alkyl, NH₂, NH-alkyl, CH₂NH₂ or OH;
   or a pharmaceutically acceptable salt thereof, is useful for the treatment or prevention of an inflammatory ophthalmic condition.

Optionally, R₁, R₂ and R₃ are independently CF₃, CONH₂, CN, halogen or NH₂.

Alkyl refers to a linear or branched alkyl group having from 1 to 10 carbon atoms, preferably from 1 to 6 carbon atoms, more preferably, from 1 to 3 carbon atoms. Preferred examples of alkyl are methyl, ethyl, n-propyl and isopropyl.

Cycloalkyl refers to a saturated or partially saturated cyclic group of from 3 to 14 carbon atoms and no ring heteroatoms and having a single ring or multiple rings including fused, bridged, and spiro ring systems, wherein the cycloalkyl is optionally substituted by one or more substituents selected from CF₃, CONH₂, CN, halogen, NH₂, NH-alkyl, alkyl, cycloalkyl or phenyl. A preferred example of cycloalkyl is cyclo-propyl.

Heterocycloalkyl refers to a saturated or partially saturated cyclic group having from 1 to 14 carbon atoms and from 1 to 6 heteroatoms selected from nitrogen, sulfur, or oxygen and includes single ring and multiple ring systems including fused, bridged, and spiro ring systems, wherein the cycloalkyl is optionally substituted by one or more substituents selected from CF₃, CONH₂, CN, halogen, NH₂, NH-alkyl, alkyl, cycloalkyl or phenyl. Preferred examples of heterocycloalkyl are piperidine, piperazine and pyrrolidine.

Embodiments of the invention that may be mentioned include those where cycloalkyl and/or heterocycloalkyl are unsubstituted.

It will be appreciated by those skilled in the art that reference herein to treatment extends to prophylaxis as well as the treatment of established conditions.

Compounds of formula (II) include some known quinolone antibiotics. Quinolone antibiotics are known to be broad spectrum antibiotics. They are chemotherapeutic bactericidal drugs and they work by preventing bacterial DNA from unwinding and duplicating. Known quinolone antibiotics include:
First-generation: cinoxacin, flumequine, oxolinic acid, piromidic acid, pipemidic acid, rosoxacin.
Second-generation: ciprofloxacin, enoxacin, fleroxacin, lomefloxacin, nadifloxacin, norfloxacin, ofloxacin, pefloxacin, rufloxacin.
Third-generation: balofloxacin, grepafloxacin, levofloxacin, pazufloxacin, sparfloxacin, temafloxacin, tosufloxacin.
Fourth-generation: clinafloxacin gatifloxacin, gemifloxacin, moxifloxacin, sitafloxacin, trovafloxacin, prulifloxacin.
In development: garenoxacin, delafloxacin.
Veterinary use: danofloxacin, difloxacin, enrofloxacin, ibafloxacin, marbofloxacin, orbifloxacin, sarafloxacin.

Compounds of formula (II) for use in the invention include (but are not limited to) known quinolone antibiotics as described above and novel compounds such as:

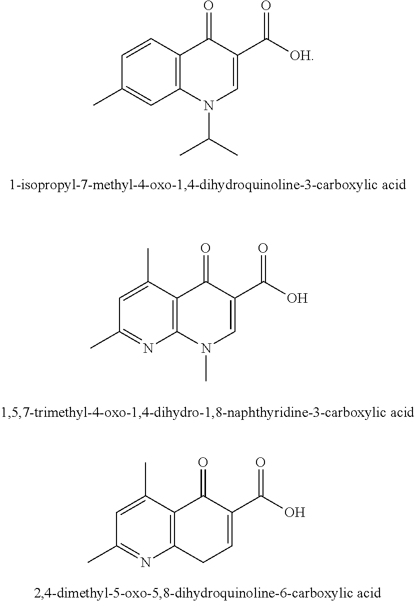 [SMILES: Cc1ccc2c(=O)c(C(=O)O)cn(C(C)C)c2c1 ; Cc1cc(C)c2c(=O)c(C(=O)O)cn(C)c2n1 ; Cc1cc(C)c2c(n1)CC=C(C(=O)O)C2=O]

It is understood that compounds for use in the invention include salts, e.g. sodium, potassium, ammonium, ethylenediamine, arginine, diethylamine, piperazine or N-Methylglucamide salts, but also extends to metabolites and pro-drugs thereof. Most aptly the free acid or salt is employed.

Compounds for use in the invention, or their pharmaceutically acceptable salts, may be chiral, and it will be understood that this invention includes any diastereomers and enantiomers of formula (II). It will also be understood that the invention includes any isotopic derivatives of the compound of formula (I) and/or formula (II).

For the avoidance of doubt, compounds of formula (I) and (II) may contain the stated atoms in any of their natural or non-natural isotopic forms. In this respect, embodiments of the invention that may be mentioned include those in which:

a) the compound of formula (I) and/or formula (II) is not isotopically enriched or labelled with respect to any atoms of the compound; and

b) the compound of formula (I) and/or formula (II) is isotopically enriched or labelled with respect to one or more atoms of the compound.

References herein to an “isotopic derivative” relate to the second of these two embodiments. In particular embodiments of the invention, the compound of formula (I) and/or formula (II) is isotopically enriched or labelled (with respect to one or more atoms of the compound) with one or more stable isotopes. Thus, the compounds of the invention that may be mentioned include, for example, compounds of formula (I) and/or formula (II) that are isotopically enriched or labelled with one or more atoms such as deuterium or the like.

Preferred examples of compounds of formula (II) include cinoxacin, flumequine, oxolinic acid, piromidic acid, pipemidic acid and rosoxacin.

Nalidixic acid or the compounds of formula (II), or their pharmaceutically acceptable salts, according to the invention are used to treat uveitis; dry eye; conjunctivitis such as allergic conjunctivitis, viral conjunctivitis, bacterial conjunctivitis and keratoconjunctivitis; ARMD; CNVM; CME; ERM; macular hole; retinopathies, including diabetic retinopathy; and as an adjunctive treatment to ophthalmic surgery.

The anti-inflammatory activity of the compounds of the invention can be demonstrated in appropriate in vitro or in vivo assays as described in the examples. Histamine (Example 1) and PGD2 (Example 2) released from IgE challenged human mast cells are both inhibited by Nalidixic acid treatment in a dose-related manner. In addition the release of Annexin-A1 (Example 3) is increased by treatment with Nalidixic acid in a dose-related manner.

The anti-inflammatory activity of the compounds of the present invention is not linked to their anti-bacterial activity and their anti-inflammatory effect can be observed at non anti-bacterial concentrations of Nalidixic acid or the analogues. Thus, according to another aspect of the invention, Nalidixic acid (I) or analogues of formula (II) or a pharmaceutically acceptable salt can be used in the treatment or prevention of inflammatory ophthalmic conditions when the amount, dose or concentration of Nalidixic Acid or analogue or salt thereof has no substantial antibiotic activity. In circumstances in which bacterial infection does not represent a component of the disease, the use of Nalidixic acid or analogue or salt thereof at sub-antibiotic doses would avoid unnecessary exposure to antibacterial activity that may lead to the generation of bacterial resistance.

According to an additional aspect of the invention, Nalidixic acid (I) or a compound of formula (II) or a pharmaceutically acceptable salt of Nalidixic acid can be used to potentiate the anti-inflammatory action of glucocorticosteroids. This activity has been demonstrated by the use of the appropriate in vitro and in vivo assays. Thus the use of a compound of the invention with steroids allows the use of traditionally sub-therapeutic, and therefore non-harmful, doses of steroids with greatly potentiated anti-inflammatory activity. Nalidixic acid or the compounds of formula (II) or a pharmaceutically acceptable salt thereof may be used according to the invention when the patient is also administered one or more glucocorticosteroids or wherein the compound of the invention is provided in combination with one or more glucocorticosteroids. Glucocorticosteroids which can be used in the invention include, but are not limited to, beclomethasone, betamethasone, budesonide, cortisone, dexamethasone, hydrocortisone, fluticasone, fluocinolone, fluromethalone, difluprednate, loteprednol, triamcinolone, meprednisone, mometasone, paramethasone and prednisolone. Particularly preferred is the use in combination with one or more of prednisolone, dexamethasone, fluocinolone, fluromethalone, difluprednate, loteprednol or triamcinolone.

Nalidixic acid, an analogue of formula (II) or a pharmaceutically acceptable salt may be used according to the invention when the patient is also administered another therapeutic agent or in combination with another therapeutic agent, wherein the therapeutic agent is selected from angiostatic peptides, such as angiostatin; angiostatic steroids, such as anecortave acetate; modulators of VEGF or FGF, such as zactima; non-steroidal anti-inflammatory drugs (NSAIDs) formulated for ocular use such as flurbiprofen, diclofenac and ketorolac; leukotriene modulators such as zilueton; anti-histamines such as cetirizine, loratidine, ketotifen and the like; antibiotics such as antibacterials, antivirals and antifungals, for example bactitracin, chloramphenicol, ciprofloxacin, fusidic acid, gentamycin, levofloxacin, neomycin alone and in combination with polymixin and gramicidin, propamide, dibromopropamide; and general cytokine/growth factor modulating agents such as cyclosporin A, phosphodiesterase inhibitors and the like. The compound of formula (1) or a salt thereof may also be administered before, during or after laser photocoagulation therapy. Laser photocoagulation therapy is used in the treatment of, for example, diabetic retinopathy and age related macular degeneration.

Nalidixic acid, an analogue of formula (II) or a salt thereof can be used to treat inflammatory conditions of the eye when administered in an amount that has antibiotic activity or in an amount than has no antibiotic activity or substantially no antibiotic activity. No substantial antibiotic activity means that the concentration of the active agent would not have clinically relevant activity on the growth of pathogenic bacteria involved in infectious ocular conditions. For susceptible bacterial strains this would be less than approximately 1 μg/ml.

The compounds described herein can be used as an anti-inflammatory agent to treat ocular inflammation. In some instances, the ocular inflammation or the ophthalmic diseases described above may be accompanied by a microbial infection of the eye. Such infection may be fungal, viral or bacterial. Nalidixic acid, an analogue of formula (II) or a salt thereof can be used to treat ocular inflammation in the presence or absence of a microbial infection. When an ocular microbial infection is present, the compounds of the invention may be administered in addition to or in combination with antibiotics. Preferred antibiotics include, but are not limited to, bactitracin, chloramphenicol, ciprofloxacin, fusidic acid, gentamycin, levofloxacin or neomycin alone or in combination with polymixin and gramicidin, propamide, di bromopropam ide.

The route of administration of Nalidixic acid, an analogue of formula (II) or a salt thereof to the eye is local. This may be topical or by intraocular injection. A preferred route of delivery is by topical administration to the eye, such as administration to the surface of the eye. Another preferred route would be by injection into the structures of the eye.

Ophthalmic pharmaceutical compositions of Nalidixic acid, an analogue of formula (II) or a pharmaceutically acceptable salt thereof represent another aspect of the invention. An injectable composition suitable for intraocular injection typically comprises a solution of the drug or a fine particle suspension, which may enable sustained delivery to the eye. Formulations are usually aqueous based and may commonly include solubilisation enhancers such as, but not limited to, polyvinyl alcohol, Tween 80 solutol, cremophore and cyclodextrin. These solubilisation enhancers may be used in combination. The formulation would typically be in the pH range of 3-8 which would be regarded as acceptable for intravitreal formulations. To achieve an acceptable pH buffering systems are sometimes used. These include but are not limited to citrate and phosphate based buffering systems. The tonicity of the intravitreal formulation may be adjusted to remain within a desirable range which typically would be 250-360 mOsm/kg. Adjustment of tonicity may be achieved for example by addition of sodium chloride. Typically intravitreal formulations are produced by sterile manufacture for single use. Preserved formulations can be used, for example formulations containing a preservative such as benzoyl alcohol. The overall volume of the injectate would normally be limited such that it is equal to or less than 0.1 ml per injection to avoid damage due to significantly increasing the volume of the vitreous humour of the eye. The dose of the active agent in the compositions of the invention will depend on the nature and degree of the condition, the age and condition of the patient and other factors known to those skilled in the art. A typical dose is 0.001-10 mg given either as a single injection with no further dosing or in multiple injections. Typically, multiple injections are given at a maximum frequency of once per week.

A topical formulation can either be an aqueous solution (eye drop), a non-aqueous solution (eye ointment) or a fine particulate suspension. Such formulations are typically made up in a manner well known to those skilled in the art. Preferred ophthalmic formulations for the topical delivery of the compounds of the invention are preservative free, however a preservative may be used. Typical preservatives include quaternary ammonium compounds such as benzylalkonium chloride or benzethonium chloride and the like; organomercurials such as phenylmercuric acetate or phenyl mercuric nitrate and the like; parahydroxybenzoates such as methylparaben, ethylparaben and the like; and chlorobutanol. Preservative agents can also act as penetration enhancers which might have the beneficial effect of increasing corneal epithelial permeability and further increasing ocular bioavailability. Tonicity and pH are important features of a topical ophthalmic formulation. In actual practice it has been found that the eye can tolerate a range of osmotic pressure values equivalent to 0.6-2% sodium chloride, without marked discomfort. In topical ophthalmic formulations EDTA or salts of EDTA are often used to modulate tonicity and also provide a preservative action. A preferred formulation has a pH close to the physiological pH of the tear duct (pH 6.5-7.5), minimising tearing and patient discomfort. However low pH is better tolerated than high pH so an acceptable pH range would be pH 4-7.5. Other agents which may be added to a topical ophthalmic formulation include viscosity modulators such as polyvinylalcohol (PVA), polyvinylpyrrolidone, methylcellulose, hydroxymethylcellulose and hydroxypropylmethylcellulose (HPMA) which increase the viscosity of the formulation. This has the advantage of minimising the drainage rate and increasing the corneal contact time.

The dose of the active agent in the compositions of the invention will depend on the nature and degree of the condition, the age and condition of the patient and other factors known to those skilled in the art. A typical dose is 0.001-100 mg given one to three times per day, for example 0.1 to 10 mg given one to three times a day.

The compositions may further comprise one or more steroids and/or another therapeutic agent. Typically, a composition comprising Nalidixic acid or a compound of formula (II) or a pharmaceutically acceptable salt thereof and one or more steroids will comprise the steroid(s) in a range of 0.001% to 5% wt/wt of the formulation. Preferably the steroid is present in a normally sub-therapeutic dose of less than 1% wt/wt of the formulation, due to the synergistic effect of the compounds of the invention as described above, although the specific dose will depend on the particular steroid used. For example, when Nalidixic acid is used, it is present within the compositions in the range of 0.001% to 5% wt/wt of the formulation and the steroid is present in a therapeutic dose of less than 1% wt/wt of the formulation.

Nalidixic acid is generally prepared through a multi-step synthetic route, which lends itself to several modifications which allow for the synthesis of Nalidixic acid analogues, such as those of formula (II):

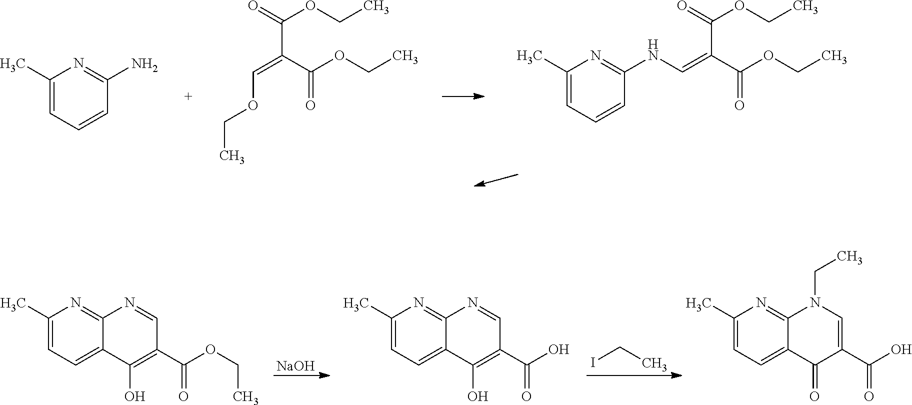

Nalidixic acid analogues of formula (II) for use in the invention may also be prepared by a multi-step synthetic procedure, as shown in the following Scheme.

The synthesis proceeds by a cyclisation starting from a di-substituted benzene or pyridine compound of general formula (III):

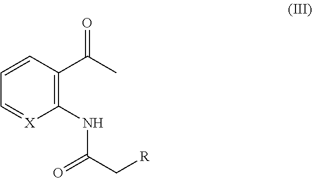

wherein R is any suitable group known to the skilled person, and X is CH or N.

The starting material is then cyclized through a Camps cyclisation to give compounds of general formula (IIIa) and (IIIb):

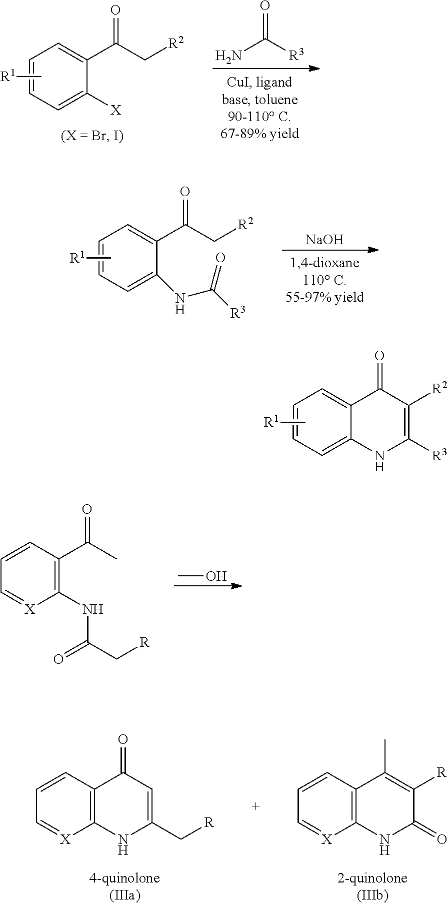

The 4-quinolone derivative of formula (IIIa) can then be isolated and further reacted to form 4-quinolone derivatives such as:

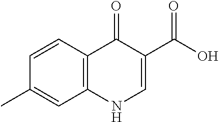

### 7-methyl-4-oxo-1,4-dihydroquinoline-3-carboxylic acid

The anti-inflammatory activity of the compounds of formula (II), or their pharmaceutically acceptable salts, can be determined by assessing their capability of inhibiting the release of histamine or PDG₂ from Human Mast Cells or promoting release of Annexin-A1

The following examples illustrate the invention

### EXAMPLES

### Example 1

### The Inhibition of Histamine Release from Human Mast Cells by Nalidixic Acid

Protocol: Human derived cord mast cells were cultured using the following method. Commercially available CD34⁺ stem cells were cultured for 2 weeks in StemSpan (StemCell Technologies, Grenoble, France) serum-free medium supplemented with 100 ng/ml human SCF, 50 ng/ml IL-6 and 1 ng/ml IL-3, and 100 μg/ml penicillin/streptomycin (Peprotech, London, UK). After eight weeks, cells were cultured in StemSpan with 10% FCS. The cells were passaged into new medium every week. Cells were used for experiments between 11 and 18 weeks following confirmation by microscopic examination, c-kit and FcRε1 staining (by FACS), of mast cell morphology. For assessment of drug effects, Nalidixic acid was incubated for 5 min with aliquots of 2×10⁵ CDMCs (cord derived mast cells) cultured in 10% FCS medium.

### Measurement of Histamine Release

A commercially-available enzyme immunoassay was used to detect and quantify histamine released in the supernatant (SPI bio, Strasbourg, France). The assay was conducted following the manufacturer's standard protocols. A standard curve ranging from 0.39-50 nM histamine was prepared using the reagent provided and the optical density was then read within 60 min in a microplate reader (at 405 nm). In some cases, the total cell content of histamine was established by freeze thawing of cells prior to challenge.

The results from these experiments are shown in FIG. 1. The data clearly demonstrates a dose related inhibition of the inflammatory mediator histamine by Nalidixic Acid.

### Example 2

### Inhibition of Prostaglandin D₂ release form Human Mast Cells by Nalidixic Acid

Human cord derived mast cells were cultured using the methodology described in Example 1.

### Measurement of PGD₂ Release

A commercially-available enzyme immunoassay (Cayman Chemical, Michigan, USA) was used to detect and quantify PGD₂ released in the supernatant. The assay was conducted following the manufacturer's standard protocols. A standard curve ranging from 78-10,000 pg/ml PGD₂ was prepared using the reagent provided and the optical density was then read within 60 min in a microplate reader (at 405 nm).

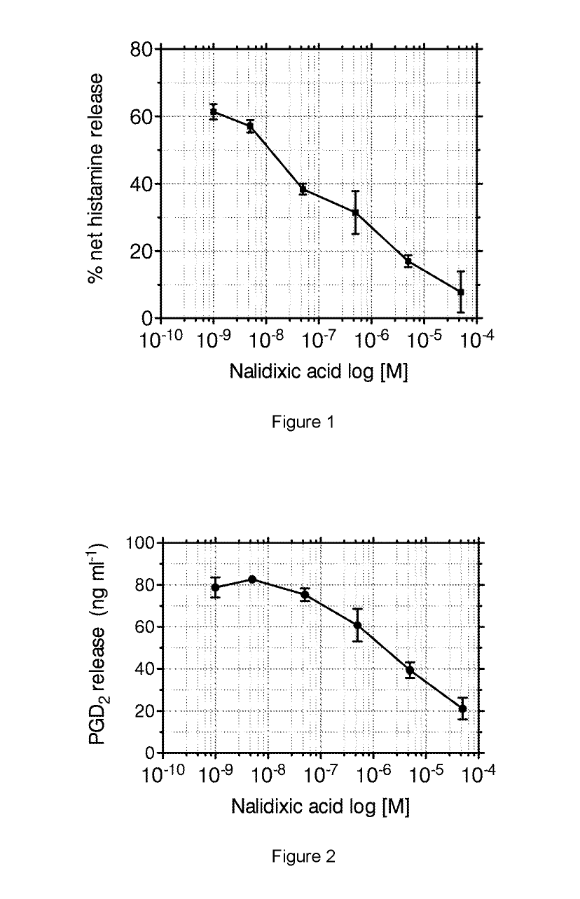

The results from these experiments are shown in FIG. 2. The data illustrates a dose related inhibition by Nalidixic acid of the inflammatory prostanoid PGD₂.

### Example 3

### Nalidixic Acid Promotes the Release of Annexin-A1 (Anx-A1) from Human Mast Cells

Human cord derived mast cells were cultured using the methodology described in Example 1.

Anx-A1 protein levels in conditioned medium were determined by ELISA. Briefly, 96-well flat-bottomed ELISA plates (Greiner, Gloucestershire, UK) were coated with 1 μg anti-Anx-A1 mAb 1B in bicarbonate buffer (pH 9.6) and incubated overnight at 4° C. After washing in the bicarbonate buffer, potentially uncoated sites were blocked with 100 μL of PBS containing 1% BSA for 1 h at room temperature. Sample aliquots (100 μL) or Anx-A1 standard solutions (prepared in 0.1% Tween-20 in PBS; concentration ranging between 10 and 0.001 μg/mL) were added for 1 h at 37° C. After extensive washing in PBS/Tween-20, 100 μL of a polyclonal rabbit anti-human Anx-A1 serum (Zymed, Invitrogen, Paisley, UK; diluted 1:1000 in PBS/Tween-20) was added (1 h at 37° C.) prior to incubation with donkey anti-rabbit 1 gG conjugated to alkaline phosphatase (1:1000; Sigma). The colour was developed by addition of 100 μL p-nitrophenyl phosphate (1 mg/mL in bicarbonate buffer, pH 9.6). Absorbance was read at 405 nm (with a 620-nm reference filter) in a microplate reader (TitertekTM, Vienna, Austria). Anx-A1 levels in the study samples were read against the standard curve and expressed as ng/ml.

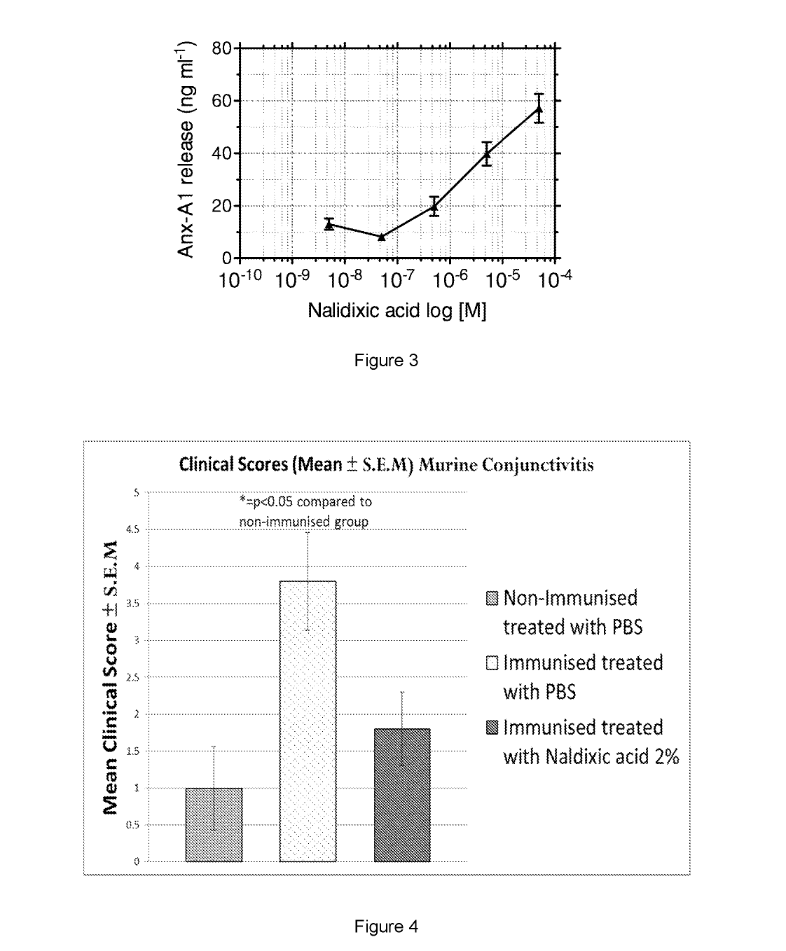

The results, as shown in FIG. 3, highlight the increase of the anti-inflammatory Annexin-Al released from human mast cells in response to increasing concentrations of Nalidixic acid.

### Example 4

### Murine Model of Allergic Conjunctivitis

Mice (Balb/C strain) were sensitised to ragweed pollen by injection of the extract mixed with alum into the hind paw. A control group was immunised with alum alone. Five animals were used in each group.

Eleven days after the initial immunisation with ragweed pollen extract the mice were challenged daily with Ragweed pollen by application to the eye (150 mg/ml antigen) and dosed twice daily (prior and after challenge with ragweed extract) with either Phosphate buffered saline (PBS, control) or 40 μl of a 2% solution of Nalidixic acid. All applications were to the left eye with the right eye acting as a control.

Conjunctivitis was assessed on the 10^{th} day 1 hour after the final application of the ragweed antigen. Assessment of the development of conjunctivitis was performed microscopically using the clinical scale shown in the table (Table 1) below. This assessment was performed by an operator unaware of the dosing protocol for the animals.

TABLE 1
Clinical scoring system for murine conjunctivitis model

| | | Score | | | |
|---|---|---|---|---|---|
| Tissue | Symptoms | 0 | 1 | 2 | 3 |
| Conjunctiva | Redness | Absent | Some or | Moderate | Severe |
| | | (same as | faint | (easily | |
| | | control) | | detectable) | |
| | Edema | Absent | Minimal but | Obvious | Severe |
| | | (same as | different to | swelling | |
| | | control) | right eye | | |
| Eyelid | Redness | Absent | Some or | Moderate | Severe |
| | | (same as | faint | (easily | |
| | | control) | | detectable) | |
| | Edema | Absent | Minimal but | Obvious | Unable |
| | | (same as | different to | swelling | to open |
| | | control) | right eye | | eye |
| Surface | Mucus | Absent | Filaments | Patches | Severe |
| | | (same as | | | |
| | | control) | | | |
| | Tears | Absent | Slight film | Glistening | Tears |
| | | (same as | | | shed |
| | | control) | | | |

Clinical scores of each group were compared statistically using the non-parametric Kruskal-Wallis test with Dunns multiple comparison test correction applied (FIG. 4). Assessment of clinical scores indicated that treatment with Nalidixic acid resulted in significant attenuation of the development of conjunctivitis, with only the immunised untreated group of animals displaying clinical signs of disease different from the unimmunised control group.

In addition, histological analysis of sections of the conjunctiva was used to assess the number of migrating eosinophils into the tissue, a key measure of the inflammatory process. No migrating cells were observed in tissue from non-immunised PBS treated (control) animals, whereas in tissue from immunised animals treated with PBS, migrating eosinophils were seen in sections from all animals (mean 3.75±0.48 S.E.M.). Treatment with Nalidixic acid resulted in the observation of no migrating eosinophils in conjunctival tissue a highly significant reduction compared to control (p<0.001). In addition histological examination of the conjunctival tissue to assess architectural changes revealed a normal well-ordered and polarised epithelial surface in the non-immunised challenge group. Whereas, repetitive challenge with ragweed and vehicle (PBS) treatment resulted in clear changes characteristic of conjunctivitis including a largely disordered cyto-architecture with many invading cells comprising but not limited to eosinophils and polymorphonuclear leukocytes.

These observations demonstrate the activity of topically applied Nalidixic acid in a murine model of allergic conjunctivitis.

### Example 5

### Murine Model of Endotoxin Induced Uveitis

The efficacy of locally applied Nalidixic acid as a potential treatment for uveitis was explored. Experimental uveitis was induced in male C57BL/6 mice (n=5 animals per group) by the intravitreal injection of the endotoxin lipopolysaccharide (LPS 0.5 ng/ml) co-injected with either vehicle (phosphate buffered saline) or Nalidixic acid at a final concentration of 0.1 μg into alternate eyes. The other eye acting as control. The inflammatory response in this model is characterised by invasion of inflammatory cells and in particular invasion of neutrophils into the retina peaks at approximately 15 hours after endotoxin treatment at which time the animals were culled. Retinas from the animals were dissected and digested into a single cell suspension. Cell numbers from retinal tissue were measured by fluorescence-activated cell sorting (FACS) analysis.

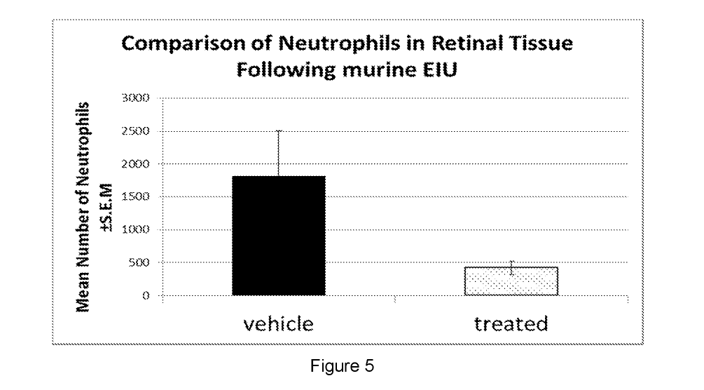

Analysis of cell infiltration following induction of uveitis with endotoxin revealed a clear reduction in invading neutrophils following co-treatment with Nalidixic acid (treated group in FIG. 5).

The ability of Nalidixic acid to attenuate neutrophil invasion, a key driver of disease, in this experiment is indicative of the potential for Nalidixic acid to treat human uveitis.

1. (canceled)

2. The method according to claim 21, wherein the one or more ophthalmic diseases is characterised by at least one of ocular inflammation, dry eye disorders, pathological ocular angiogenesis, or retinal or sub-retinal edema.

3. The method according to claim 21, wherein the one or more ophthalmic diseases is conjunctivitis.

4. The method according to claim 21, wherein the one or more ophthalmic diseases is dry eye.

5. The method according to claim 21, wherein the one or more ophthalmic diseases is inflammation or dry eye caused by refractive eye surgery.

6. The method according to claim 21, wherein the one or more ophthalmic diseases is uveitis.

7. The method according to claim 21, wherein the one or more ophthalmic diseases is age related macular degeneration (ARMD).

8. The method according to claim 21, wherein the one or more ophthalmic diseases is diabetic retinopathy.

9. The method according to claim 21, wherein the one or more ophthalmic diseases is choroidal neovascular membrane (CNVM), cystoid macular edema (CME), epiretinal membrane (ERM), or macular hole.

10. The method according to claim 21, wherein the compound of formula (I), the compound of formula (II), or the pharmaceutically acceptable salt of formula (I) or formula (II) is formulated for topical application to the eye.

11. The method according to claim 21, wherein the compound of formula (I), the compound of formula (II), or the pharmaceutically acceptable salt of formula (I) or formula (II) is formulated for intraocular injection.

12. The method according to claim 21, further comprising administering.

13. The method according to claim 21, further comprising administering to the patient at least one therapeutic agent selected from angiostatic peptides; angiostatic steroids; modulators of VEGF or FGF; non-steroidal anti-inflammatory drugs (NSAIDs) formulated for ocular use; glucocorticosteroids; leukotriene modulators; anti-histamines; and general cytokine/growth factor modulating agents.

14. The method according to claim 21, wherein the step of administering occurs before, during, or after laser photocoagulation therapy.

15. The method according to claim 12, wherein [i] the compound of formula (I), the compound of formula (II), or the pharmaceutically acceptable salt of formula (I) or formula (II), and [ii] the one or more glucocorticosteroids are administered in combination.

16. A pharmaceutical composition comprising:
Nalidixic acid of formula (I), an analogue, or a pharmaceutically acceptable salt thereof, 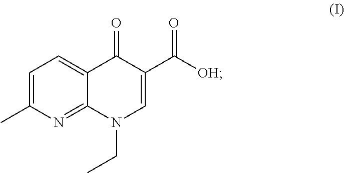 [SMILES: CCn1cc(C(=O)O)c(=O)c2ccc(C)nc21]
and
at least one of a diluent or a carrier.

17. (canceled)

18. The pharmaceutical composition according to claim 16, wherein the pharmaceutical composition is suitable for topical delivery to the eye or for intraocular injection.

19. The method according to claim 21, wherein the compound is Nalidixic acid of formula (I) or a pharmaceutically acceptable salt thereof.

20. The pharmaceutical composition according to claim 16, wherein the Nalidixic acid analogue is a compound of formula (II); 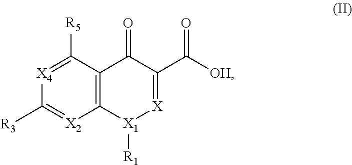
wherein,
X and X₁ independently represent CH or N;
X₂ represents C(R₂) or N;
X₄ represents C(R₄) or N;
R₁ is H, CF₃, CONH₂, CN, halogen, NH₂, NH-alkyl, alkyl, cycloalkyl, or phenyl, and is optionally substituted with one or more R₆; wherein R₁ may form part of a cycle with R₂;
R₂ is H, CF₃, CONH₂, CN, halogen, NH₂, alkyl, O-alkyl, or S-alkyl; wherein R₂ may form part of a cycle with R₁;
R₃ is H, CF₃, CONH₂, CN, halogen, NH₂, alkyl, O-alkyl, pyridyl, cycloalkyl, or heterocycloalkyl and is optionally substituted with one or more R₆; wherein R₃ may form part of a cycle with R₄;
R₄ is H, F, or O-alkyl; wherein R₄ may form part of a cycle with R₃;
R₅ is H, F, Cl, alkyl, O-alkyl, or NH₂;
R₆ is F, alkyl, NH₂, NH-alkyl, CH₂NH₂, or OH;
or a pharmaceutically acceptable salt thereof.

21. A method for treating or preventing one or more inflammatory ophthalmic diseases, the method comprising administering locally to a patient a compound of Nalidixic acid of formula (I) or a Nalidixic acid analogue of formula (II): 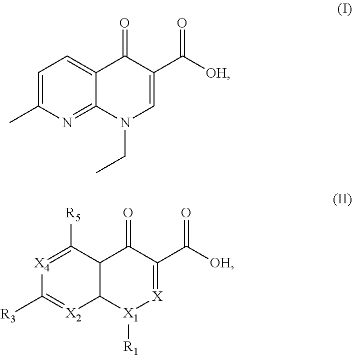
wherein,
X and X₁ independently represent CH or N;
X₂ represents C(R₂) or N;
X₄ represents C(R₄) or N;
R₁ is H, CF₃, CONH₂, CN, halogen, NH₂, NH-alkyl, alkyl, cycloalkyl, or phenyl, and is optionally substituted with one or more R₆; wherein R₁ may form part of a cycle with R₂;
R₂ is H, CF₃, CONH₂, CN, halogen, NH₂, alkyl, O-alkyl, or S-alkyl; wherein R₂ may form part of a cycle with R1;
R₃ is H, CF₃, CONH₂, CN, halogen, NH₂, alkyl, O-alkyl, pyridyl, cycloalkyl, or heterocycloalkyl and is optionally substituted with one or more R₆; wherein R₃ may form part of a cycle with R₄;
R₄ is H, F, or O-alkyl; wherein R₄ may form part of a cycle with R₃;
R₅ is H, F, Cl, alkyl, O-alkyl, or NH₂;
R₆ is F, alkyl, NH₂, NH-alkyl, CH₂NH₂, or OH;
or a pharmaceutically acceptable salt thereof.

22. The method according to claim 21, wherein an amount of the compound of formula (I), the compound of formula (II), or the pharmaceutically acceptable salt of formula (I) or formula (II) that is administered to the patient has no substantial antibacterial activity.

23. The method according to claim 13, wherein [i] the compound of formula (I), the compound of formula (II), or the pharmaceutically acceptable salt of formula (I) or formula (II), and [ii] the at least one therapeutic agent are administered in combination.
